(19) United States
(12) Patent Application Publication
(10) Pub. No.: US 2022/0000391 A1
(43) Pub. Date: Jan. 6, 2022

(54) METHODS AND DEVICES FOR COLLECTION OF EXHALED AEROSOLS

(71) Applicant: iREPERTOIRE, INC., Huntsville, AL (US)

(72) Inventors: Jian Han, Humtsville, AL (US); Wenjing Pan, Huntsville, AL (US)

(21) Appl. No.: 16/919,015

(22) Filed: Jul. 1, 2020

(51) Int. Cl. A61B 5/097; A61B 5/08; A61B 10/00

(52) U.S. Cl. CPC A61B 5/097; C12Q 1/686; A61B 10/0051; A61B 5/082

(57) ABSTRACT

The present disclosure relates to methods and devices for the collection of exhaled aerosols. In some embodiments, the device disclosed herein comprises a cup and an attachable lid, wherein the cup has a conical base and the lid includes a pipette access port with a removable cap and a sealed buffer reservoir. In some embodiments, the method for a subject to collect exhaled aerosols disclosed herein comprises the steps of placing a collection cup against the subject's mouth, the subject coughing into the collection cup at least once, immediately closing the device by reattached a lid to the collection cup, placing the collection cup in a transport bag, and providing the transport bag to a processor. The processor can then incubate the transport bag containing the collection cup, add buffer to the collection cup to rinse and suspend any collected exhaled aerosols, and then extract the buffer containing the exhaled aerosols either for storage or for immediate amplification and sequencing for the detection of pathogens.

### BACKGROUND OF THE INVENTION

Traditional methods and devices for the detection of respiratory illnesses have involved the use of nasal swabs. The rationale behind use of such swabs is the belief that they are effective by reaching deep into the nasopharyngeal cavity, where the pathogen for the illness is located. Sputum samples, in contrast, are typically not thought to work well for this purpose, because oral and nasal cavities are heavily-guarded by the immune system such that the presence of concentrated enzymes and antibodies degrade and clear such pathogens, making detection difficult.

The global outbreak of severe acute respiratory syndrome coronavirus 2 (SARS-CoV-2), termed COVID-19, in 2020 has highlighted the use of nasal swabs in the detection of disease. In the United States alone, several million nasal swab COVID-19 tests were performed in the first few months of the pandemic. Even this number, however, is not sufficient. It is estimated that a test volume of 5-20 million tests per day may be required to reopen the U.S. economy safely, which is not feasible using traditional swab testing. Three bottlenecks restricting the access of tests include: (1) a limited number of qualified labs and trained personnel to run the tests; (2) a limited supply of nasopharyngeal sampling kits and a shortage of critical test reagents (for RNA extraction); and (3) limited access to healthcare workers and trained professionals qualified to take the samples using the swabs.

Swab testing is not an ideal sampling approach for pandemic management for the following reasons: (1) the nasopharyngeal swab sampling is not really a “noninvasive” procedure; it is nicknamed “the brain swab” because it needs to reach very deep into the nasopharyngeal cavity, causing pain and discomfort; (2) only a nurse practitioner or a trained healthcare worker can perform the sampling task at a healthcare institution; and (3) the collected viral sample needs to be sent to a central lab in a “viral transfer buffer” (high concentration of unique salt to stabilize the viral RNA). Once the sample arrives in the lab, an extraction step is needed to recover the viral RNA and remove the salt before a molecular assay (PCR, multiplex PCR, or isothermal amplification) can be performed. Both the transfer buffer and the extraction reagents are in severe shortage due to the current demand. As such, a strong need exists for more robust, less invasive, and more readily-sourced sample collection methods and devices.

### SUMMARY OF THE INVENTION

In some embodiments, the present disclosure relates to a device for the collection of exhaled aerosols comprising: a collection cup, wherein the exterior base of the collection cup is flat; and a lid, wherein the lid comprises a port that forms a channel through the lid and wherein the lid is configured to attach to the collection cup. In some embodiments, the device is cylindrical. In some embodiments, the interior base of the collection cup is conical, wherein the interior base narrows as it approaches the base of the collection cup. In some embodiments, the collection cup and lid are threaded to allow the lid to screw onto and unscrew from the rim of the collection cup. In some embodiments, the port is circular and the channel is cylindrical. In some embodiments, the port and the channel are hexagonal. In some embodiments, the port and the channel are approximately 25 mm in diameter.

In some embodiments of the device, the channel comprises a watertight top seal and bottom seal. In some embodiments, the top seal comprises a rubber gasket and the bottom seal comprises an adhesive aluminum foil seal. In some embodiments, a portion of the top seal is attached to the lid. In some embodiments, the top seal is comprised of plastic. In some embodiments, the channel comprises a buffer. In some embodiments, the device further comprises a label. In some embodiments, the interior surface of the collection cup is coated with RNAase inhibitor. In some embodiments, the interior of the collection cup is partially coated with a colorimetric or physical indicator of the presence of exhaled aerosols.

In certain embodiments, the present disclosure relates to an exhaled aerosol collection kit, comprising: a device comprising: a collection cup, wherein the exterior base of the collection cup is flat and interior of the collection cup is coated with RNAase inhibitor; and a lid, wherein the lid comprises a port that forms a channel through the lid and wherein the lid is configured to attach to the collection cup; a barcode; a transport bag; and a transport box. In some embodiments of the kit, the channel comprises a top seal, a bottom seal, and a buffer.

In certain embodiments, the present disclosure relates to a method of using the kit disclosed herein by a subject, comprising the steps of: scanning the barcode to associate the device with the subject's software account; removing the lid from the collection cup; holding the subject's breath for up to 15 seconds; placing the collection cup over the subject's mouth and coughing into the collection cup at least once; closing the device by reattaching the lid to the collection cup; placing the device in a transport bag; sealing the transport bag; placing the transport bag in a transport box; sealing the transport box; and providing the transport box to a testing facility.

In some embodiments of the method, the subject coughs 3 to 5 times into the collection cup. In some embodiments, the subject spits into the collection cup at least once. In some embodiments, the subject spits into the collection cup at least once in lieu of coughing. In some embodiments of the method, the method further comprises the step of the subject wrapping a tissue around the rim of the collection cup when performing the step of coughing into the collection cup. In some embodiments of the method, the method further comprises processing the device by the testing facility, comprising the additional steps of: removing the transport bag from the transport box; incubating the transport bag containing the device at approximately 65° C. for approximately 30 minutes; adding buffer to the collection cup; sealing the lid on the collection cup; agitating the device; extracting buffer from the collection cup; and subjecting the extracted buffer to PCR amplification using primers specific to at least one pathogen to form an amplicon; sequencing the amplicon; and uploading the sequencing results to the subject's software account. In some embodiments of the method, the sequencing results are formatted to show the presence or absence of the at least one pathogen in the subject's exhaled aerosols.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 is an exploded view of an embodiment of the exhaled aerosol collection device disclosed herein.

FIG. 2 depicts an embodiment of the lid of an exhaled aerosol collection device. (A) A view of the bottom of the lid. (B) A view of the top of the lid.

FIG. 3 depicts an alternative embodiment of an exhaled aerosol collection device. (A) An isometric exterior view of the device. (B) An isometric transparent view of the device.

### DETAILED DESCRIPTION

The present disclosure relates to methods and devices for the collection of exhaled aerosols. In some embodiments, the device disclosed herein comprises a cup and an attachable lid, wherein the cup has a conical base and the lid includes a pipette access port with a removable cap and a sealed buffer reservoir. In some embodiments, the method for a subject to collect exhaled aerosols disclosed herein comprises the steps of placing a collection cup against the subject's mouth, the subject coughing into the collection cup at least once, immediately closing the device by reattaching a lid to the collection cup, placing the collection cup in a transport bag, and providing the transport bag to a processor. The processor can then incubate the transport bag containing the collection cup, add buffer to the collection cup to rinse and suspend any collected exhaled aerosols, and then extract the buffer containing the exhaled aerosols either for storage or for immediate amplification and sequencing for the detection of pathogens.

The term “barcode” as used herein means a barcode, QR code, or other computer-readable code.

The term “buffer” as used herein means lysis-equilibration-wash (LEW) buffer or other suitable buffer.

The term “exhaled aerosols” as used herein refers to droplets of mucous and other biological materials emitted from a subject's respiratory tract by sneezing, coughing, talking or breathing. Such materials may include pathogens, such as bacteria or viruses from a subject. Viruses may include, but are not limited to, coronavirus, rhinoviruses, influenza, respiratory syncytial virus, measles, mumps and adenovirus.

The term “sanitary gloves” as used herein means latex gloves, nitrile gloves, vinyl gloves, or other gloves suitable for use as personal protective equipment (PPE).

The term “subject” as used herein means an individual from whom exhaled aerosols are collected using a device disclosed herein.

The term “testing facility” as used herein means a laboratory with personnel and equipment sufficient to perform the processing steps for the device as disclosed herein. Examples of testing facilities include, but are not limited to, hospital labs, university core labs, and private testing labs.

The term “transport bag” as used herein means a bag suitable for the transport of the device containing exhaled aerosols under applicable law. The transport bag may be a biohazard bag.

The applicants observed the transmission of respiratory disease, such as COVID-19, through aerosols produced by infected individuals from sneezing, coughing and even talking and posited that, due to their transmission of disease, such aerosols contain pathogens. In lieu of the conventional, invasive nasal swab testing, the applicants have developed non-invasive, low-cost, and user-friendly sample collection methods and devices to capture exhaled airborne droplets that may contain pathogens from infected individuals for purposes of detection of pathogens, such as COVID-19, as disclosed herein.

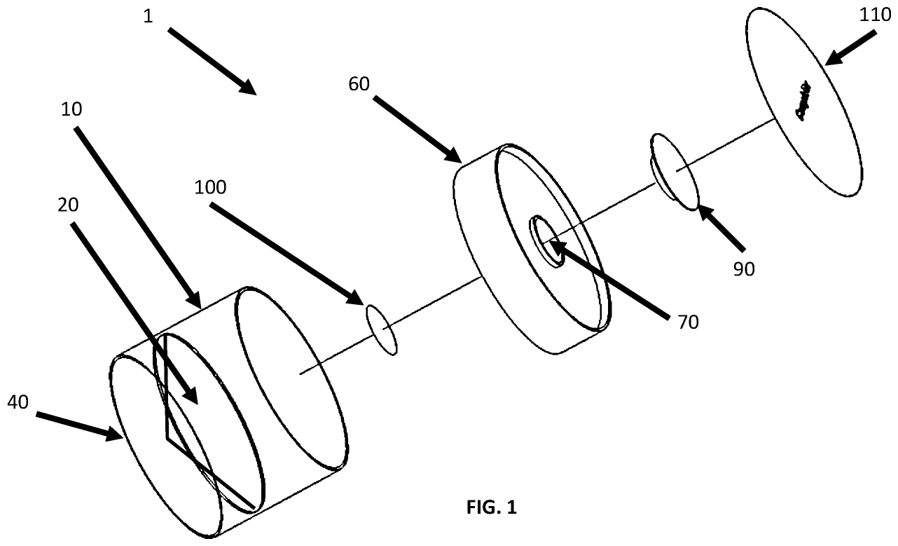

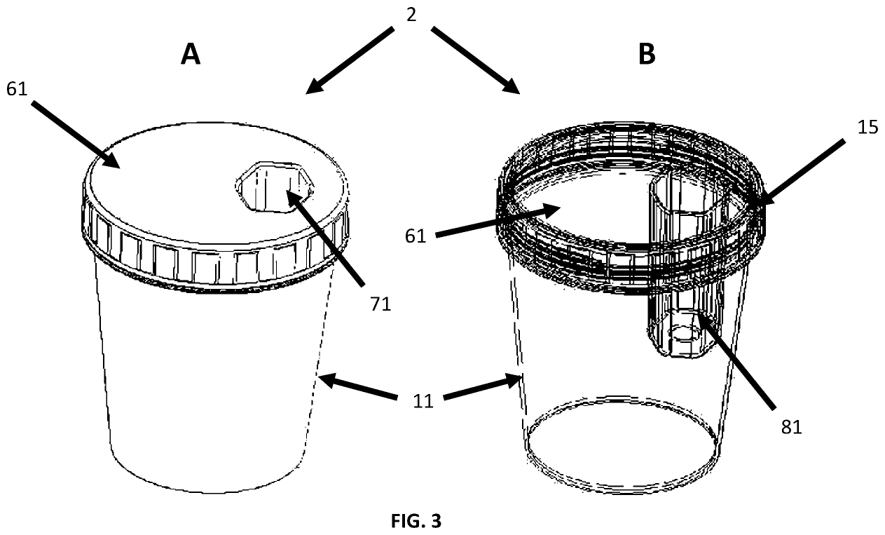

Disclosed herein are devices for collecting exhaled aerosols. These devices may be used with the methods disclosed herein. In one example of the device, as shown in FIG. 1, the device 1 comprises a collection cup 10 and a lid 60. In some embodiments, the device 1 is cylindrical. Preferably, the embodiments of the collection cup and lid are made from plastic, although other materials suitable for biological sample collection can be used. The interior base 20 of the collection cup 10 can be conical, as shown in FIG. 1, wherein the cone narrows as it approaches the exterior base 40 of the collection cup 10 so that any fluid can concentrate at the bottom due to gravity. Alternatively, in other embodiments the interior base of the collection cup can be flat, curved, or angled. The exterior base of the collection cup is preferably flat to maintain stability. In each embodiment, the lid is configured to affix to the collection cup. The lid 60 can be configured to snugly fit on the exterior of the collection cup 10 by friction, as shown in FIG. 1. Alternatively, as shown in FIG. 3, the collection cup 11 and lid 61 can be threaded to allow the lid 61 to be fastened or loosened from the rim 15 of the cup 11. Alternatively, other means of attaching and de-attaching the lid to the collection cup can be used, as known to those of skill in the art.

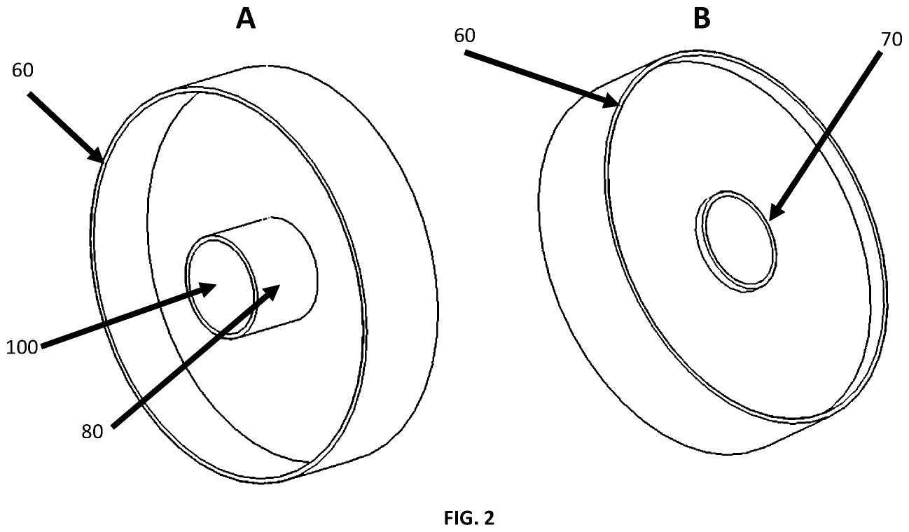

As shown in FIG. 2, the lid 60 can include a port 70 that forms an open channel 80 through the lid 60. The port 70 can be centered on the lid 60. Alternatively, as shown in FIG. 3, the port 71 can be offset from the center of the lid 61. The channel 80 can extend away from the top of the lid 60 toward the bottom of the lid, as shown in FIG. 2. In some embodiments, however, the channel 81 can extend outward beyond the bottom of the lid 61, as shown in FIG. 3. The port 70 and channel 80 are preferably circular, as shown in FIG. 2, although other shapes, including but not limited to hexagonal, as shown in FIG. 3, can be used. In each embodiment, the port can be configured to be wide enough for a pipette, or pipette tip, to pass through the port. In FIG. 3, for example, the port 71 and channel 81 are approximately 25 mm in diameter.

As shown in FIG. 1, the channel 80 can further include a top seal 90 and/or a bottom seal 100. A seal can be reusable, for example a rubber gasket shaped to fit into and form a water-tight friction seal in the port 70 as shown by the top seal 90 in FIG. 1, or it can be configured to be punctured or removed, for example an adhesive aluminum foil seal that can be punctured with a pipette tip as shown by the bottom seal 100 in FIG. 1. In some embodiments, a portion of the top seal is attached to the lid. In some embodiments, the top seal is comprised of plastic. In some embodiments, the channel formed by the port can comprise a buffer. The buffer can be LEW buffer. Each embodiment of the device can further include a label. In some embodiments, such as the embodiment shown in FIG. 1, the label 110 is located on the top of the lid 60. The label can be made of paper or other material on which product and sample information can be printed. The label can be placed around or cover the port. The label may include a barcode or an alphanumeric code to identify the particular device. In other embodiments, a barcode or alphanumeric code is provided on a separate sheet provided with the device.

The device may also include a physical or chemical indicator to ensure that a sufficient sample of exhaled aerosols has been collected from a subject. Such indicator serves as a positive control that can be assessed visually. For example, the inner wall or interior base of the collection cup may be lined, in part, with evenly-distributed crystallization spots or a colorimetric indicator to allow visual confirmation that exhaled aerosols have been deposited in the collection cup. A visual guide showing the recommended appearance of the indicator can be included to assist a subject. The interior surfaces of the device may also be coated with RNAase inhibitor or other material designed to inhibit degradation of viral particles.

The device may be packaged as a kit, comprising a transport bag, a pair of sanitary gloves, an alcohol wipe, at least one form, and a transport box. The form may include a barcode or alphanumeric code with which the device is specifically associated. The form may also include other fields for the subject to complete including, but not limited to, the subject's name, the subject's gender, the subject's age, the date and time the device is used by the subject, the subject's contact information, the subject's account name associated with a software application used in connection with the device, and other relevant information. The transport box may be a cardboard mailing box that is large enough to house the transport bag, device and form. The transport box may be pre-labeled with suitable labels for shipping a biological sample as required under applicable law.

Also disclosed herein are methods for collecting exhaled aerosols from a subject. The method may comprise the steps of the subject removing the lid of a collection cup of a device disclosed herein, the subject holding their breath, the subject coughing into the collection cup at least once, closing the device by reattaching the lid to the collection cup, placing the device in a transport bag, sealing the transport bag, and sending the transport bag containing the device to a testing facility. The step of the subject coughing into the collection cup is preferably performed while the collection cup is placed over the subject's mouth. A tissue may be wrapped around the lid of the collection cup to mitigate the escape of any exhaled aerosols while the subject coughs. In some embodiments, the subject holds their breath for at least five seconds. In other embodiments, the subject holds their breath for up to approximately fifteen seconds. In some embodiments, the subject coughs into the collection cup three to five times. The transport bag may be configured to comprise an adhesive seal proximal to the opening of the transport bag, wherein the adhesive seal includes a removable backing such that removal of the backing exposes the adhesive, which may be folded over to seal the transport bag. Other means of sealing the transport bag may be used as known to those of skill in the art.

The subject may wash their hands prior to the step of removing the lid of the collection cup. The subject may also wear sanitary gloves prior to the step of removing the lid of the collection cup. The exterior surface of the device may be cleaned after the step of closing the device. The cleaning may be performed using alcohol wipes, by rinsing the device and allowing it to dry, or other suitable method for disinfection.

A subject may download a software application and form an account to link the subject with the device that the subject uses. The software application may be a mobile application. The software application may be configured to allow entry of a subject's information. The software application may be configured to scan a barcode accompanying a device using a smart phone camera, if used on a mobile device, or using a peripheral camera, if used on a personal computer.

The device can then be processed by a testing facility. The method may comprise the steps of: removing the transport bag from the transport box; incubating the transport bag containing the device at approximately 65° C. for approximately 30 minutes; recording the information contained on the form; adding approximately 2 mL buffer to the collection cup; resealing the device and ensuring the lid is tightly affixed to the collection cup; agitating the device; and extracting buffer from the collection cup. In other embodiments, different volumes of buffer may be used. In certain embodiments, such as shown in FIG. 2, the buffer may be added to the collection cup 10 by removing the top seal 90 from the lid 60 and piercing the bottom seal 100 with a pipette or other suitable instrument, allowing buffer contained within the channel 80 to fall into the collection cup 10. Alternatively, in each embodiment the buffer may be added by removing the lid and pipetting the buffer into the collection cup, in which case the step of resealing the lid comprises ensuring the lid is tightly affixed to the collection cup. To concentrate the buffer on the bottom of the collection cup, an additional step of tapping the device on a surface or allowing the collection cup to rest for a period of time may be performed after agitating the device and before extracting buffer from the collection cup. If the collection cup is allowed to rest, it may be allowed to rest for up to five minutes, although other durations are possible including, but not limited to, up to thirty minutes.

The testing facility may further store the device at −80° C. for future testing. The extracted buffer may be subjected to amplification, including but not limited to realtime PCR, using primers specific for at least one pathogen of interest, and sequencing steps to assay the exhaled aerosols within the buffer for the presence of the at least one pathogen of interest. The results of the sequencing assay may be uploaded to the account associated with the subject, allowing the subject that owns the account to check the testing results for the presence or absence of the at least one pathogen of interest.

Compared to conventional swab testing, the methods and devices disclosed herein have several advantages. The methods and devices disclosed herein are inexpensive, the required materials are relatively easy to source, the test is user-friendly and can be performed by the subject, healthcare workers are not required and therefore will not be exposed to potential pathogens from the subject, and the subject can obtain their testing results remotely using a software application. Greater testing coverage compared to conventional methods, whether due to reduced cost, ease of use, and/or removing the need for trained health care workers to perform the test, will result in fewer missed positive tests by using the devices and methods disclosed herein. Although the present invention and its advantages have been described in detail, it should be understood that various changes, substitutions and alterations can be made herein without departing from the spirit and scope of the invention as defined in the appended claims.

The following examples, applications, descriptions and content are exemplary and explanatory, and are non-limiting and non-restrictive in any way.

### Examples

### Sample Collection and Processing for COVID-19 Detection

The following procedures are one embodiment of the methods disclosed herein of collecting exhaled aerosols from a subject and processing the device.

### Exhaled Aerosol Collection Kit Contents

1. COVID-19 Lab Requisition Form

2. Transport Bag

3. Collection Cup

4. One pair of gloves

5. EtOH wipe

6. Transport Box

### Exhaled Aerosol Collection: Cough Method

1. Wash hands with running water and hand soap for 2 mins.

2. Wear provided gloves for the duration of the following procedure.

3. Open the lid of the collection cup.

4. Take a deep breath and hold the breath for up to 15 seconds, cover the mouth with the collection cup and cough into the collection cup 5 times.
Note: Dry cough is preferred; do not spit sputum, phlegm or saliva into the collection cup. Viral particles will stick on the interior surface of the collection cup. In an alternative method, however, this step may further comprise the steps of: using your tongue to massage the sides of your mouth and spitting a small amount of saliva into the bottom of the collection cup.

5. Turn the lid slowly and firmly to make sure the lid is secure.

6. Rinse the exterior surface of the device and let it dry.

7. Clean the exterior surface of the collection cup with an EtOH wipe.

8. Please the device in the pocket of the transport bag.

9. Remove adhesive backing and fold over to overlap the pocket and secure the adhesive to the transport bag.

10. Remove gloves, wash hands thoroughly, place the transport bag in the transport box, and close the transport box.

11. Contact a certified sample collecting service provider using the CoughDX app and transfer the transport box containing the device to a local test lab. The exhaled aerosols in the device can remain at room temperature for 12 hours or at 4° C. for 72 hours.

### Device Processing in the Testing Lab

1. Spray the transport box with bleach and clean the transport box thoroughly.

2. Open the transport box and take out the transport bag. Spray the transport bag with bleach and clean the transport bag thoroughly.

3. Open the transport bag and take out the device. Spray the exterior of the device with bleach and clean the exterior of the device thoroughly.
Alternatively: Rinse the collection cup and lid with 0.5% Tween20 in NF water for 5 min; rinse the collection cup and lid with NF water for 5 min; and incubate the collection cup and lid at 65° C. for 1 hour.

4. Scan the barcode on the label of the lid to acquire the subject's software account and subject's information.

5. Open the lid carefully, add 1.5 mL LEW buffer into the collection cup and resecure the lid on the collection cup. The LEW buffer comprises 70% EtOH (LEW buffer #1) or Saline solution (LEW buffer #2).

6. Vortex the device for approximately 10 seconds, and gently tap the device on a surface approximately 10 times to concentrate the LEW buffer to the bottom of the collection cup.

7. Take an appropriate volume of LEW buffer (see realtime PCR method disclosed below) for amplification.

8. Spray bleach inside and outside of the device and dispose of the device.

### Realtime PCR for COVID-19 Detection

(1) Material
a. Bio-Rad Reliance One-Step Multiplex Supermix (Cat #12010176)
b. Hard-Shell Thin-Wall 96-Well skirted PCR plate (Cat # HSP 9655)
c. Microseal ‘B’ adhesive seals (Cat # MSB1001)
d. Primers in Table 1.

TABLE 1
2019-Novel Coronavirus (2019-nCoV) Real-time
rRT-PCR Panel Primers and Probes

| | | Oligonucleotide | Working |
|---|---|---|---|
| Name | Description | Sequence (5′ > 3′) | Conc. |
| 2019-nCoV_N1-F | 2019-nCoV_N1 | 5′-GAC CCC AAA ATC AGC | 20 μM |
| | Forward Primer | GAA AT-3′ | |
| 2019-nCoV_N1-R | 2019-nCoV_N1 | 5′-TCT GGT TAC TGC CAG | 20 μM |
| | Reverse Primer | TTG AAT CTG-3′ | |
| 2019-nCoV_N2-F | 2019-nCoV_N2 | 5′-TTA CAA ACA TTG GCC | 20 μM |
| | Forward Primer | GCA AA-3′ | |
| 2019-nCoV_N2-R | 2019-nCoV_N2 | 5′-GCG CGA CAT TCC GAA | 20 μM |
| | Reverse Primer | GAA-3′ | |
| 2019-nCoV_N3-F | 2019-nCoV_N3 | 5′-GGG AGC CTT GAA TAC | 20 μM |
| | Forward Primer | ACC AAA A-3′ | |
| 2019-nCoV_N3-R | 2019-nCoV_N3 | 5′-TGT AGC ACG ATT GCA | 20 μM |
| | Reverse Primer | GCA TTG-3′ | |
| RP-F | RNAse P | 5′-AGA TTT GGA CCT GCG | 20 μM |
| | Forward Primer | AGC G-3′ | |
| RP-R | RNAse P | 5′-GAG CGG CTG TCT CCA | 20 μM |
| | Reverse Primer | CAA GT-3′ | |

(2) Equipment
a. Bio-Rad CFX96 Touch Real-Time PCR detection system.
b. Pipette
c. Pipette tips

(3) Sample Preparation
a. For LEW buffer #1: Take 50 uL LEW buffer from cup and add in PCR tube, incubate PCR tube at 95 C for 30 mins with lid open until all the liquid evaporated.
b. For LEW buffer #2: Take 13 uL LEW buffer from cup and add in PCR tube for real-time PCR.

(4) Setup Real-Time PCR

a. Prepare a Master Mix with the 4× Reliance Supermix and primers. Record the lot information and initial as each component is added below according to Table 2. Vortex the final prepared Master Mix.

TABLE 2

| | LEW1 Volume/ | LEW 2 Volume/ |
|---|---|---|
| Reagent | Reaction (μL) | Reaction (μL) |
| NFH20 | 13 | 0 |
| 4X Reliance Supermix | 5 | 5 |
| CDC Forward Primer (5 pmol/μL) | 1 | 1 |
| CDC Reverse Primer (5 pmol/μL) | 1 | 1 |
| RNA template | 0 | 13 |
| Total | 20 | 20 |

b. Run the real-time PCR with the conditions listed in Table 3.

TABLE 3

| Step | Cycles | Temp | Time |
|---|---|---|---|
| Reverse transcription | 1 | 50° C. | 10 min |
| Enzyme activation | 1 | 95° C. | 10 min |
| Amplification | 45 | 95° C. | 10 sec |
| | | 60° C. | 30 sec |

### Results

The results from nine tests performed according to the above methods produced the results shown in Table 4.

TABLE 4

| | CDC | | | |
|---|---|---|---|---|
| Test | Target | Cq | Efficiency | Efficiency R² |
| 1 | N1 | 36.04 | 0.796 | 0.9994 |
| 2 | N1 | 36.85 | 0.807 | 0.9994 |
| 3 | N2 | 35.18 | 0.769 | 0.9991 |
| 4 | N2 | 35.53 | 0.787 | 0.9992 |
| 5 | N3 | 36.18 | 0.813 | 0.9993 |
| 6 | N3 | 35.09 | 0.801 | 0.9987 |
| 7 | RP | 36.48 | 0.825 | 0.9993 |
| 8 | RP | 37.40 | 0.812 | 0.9993 |
| 9 | N1 | 34.20 | 0.814 | 0.9993 |

Comparison of Disclosed Methods with Conventional Nasal Swab Testing in an Individual Diagnosed with COVID-19

To compare the efficacy of the disclosed methods to detect pathogens compared to the conventional nasal swab testing method, the applicants tested an individual with a confirmed positive test for COVID-19. The applicants tested the individual using: (1) the disclosed method wherein the individual spat into the collection cup; (2) the disclosed method wherein the individual spat and coughed into the collection cup; and (3) the traditional nasal swab method using a Puritan® nasal swab. Two sample volumes were tested on each of three target COVID-19 sequences using each of the three tests listed in (1), (2) and (3) above. As shown in Table 5, all three methods detected the presence of a COVID-19 specific sequence. N1 and N2 represent target sequences specific for COVID-19. RP represents a control sequence not specific to COVID-19.

TABLE 5

| | | |
|---|---|---|
| Disclosed Method | Disclosed Method | |
| (Spit Only) | (Cough + Spit) | Nasal Swab |

System

| | | | | |
|---|---|---|---|---|
| 2 mL | 2 mL | 2 mL | 2 mL | 1 mL |

Direct Input

| | 13 uL | 6.5 uL | 13 uL | 6.5 uL | 5 uL |
|---|---|---|---|---|---|
| | Average Cq value | | | | |
| N1 | 34.2 | 35.6 | 19.2 | 21.5 | 15.3 |
| N2 | 35.0 | 36.4 | 20.1 | 23.0 | 16.7 |
| RP | 25.6 | 26.5 | 26.3 | 27.2 | 25.0 |
| | Working Percentage | | | | |
| N1 | 100% | 100% | 100% | 100% | 100% |
| N2 | 100% | 100% | 100% | 100% | 100% |
| RP | 100% | 100% | 100% | 100% | 100% |
| | Working Count | | | | |
| N1 | 3 | 3 | 3 | 3 | 3 |
| N2 | 3 | 3 | 3 | 3 | 3 |
| RP | 2 | 2 | 2 | 2 | 2 |

The methodologies, devices, and the various embodiments thereof described herein are exemplary. Various other embodiments of the methodologies and devices described herein are possible.

Now, therefore, the following is claimed:

1. A device for the collection of exhaled aerosols comprising:
a collection cup, wherein the exterior base of the collection cup is flat; and
a lid, wherein the lid comprises a port that forms a channel through the lid and wherein the lid is configured to attach to the collection cup.

2. The device of claim 1, wherein the device is cylindrical.

3. The device of claim 1, wherein the interior base of the collection cup is conical, wherein the interior base narrows as it approaches the base of the collection cup.

4. The device of claim 1, wherein the collection cup and lid are threaded to allow the lid to screw onto and unscrew from the rim of the collection cup.

5. The device of claim 1, wherein the port is circular and the channel is cylindrical.

6. The device of claim 1, wherein the port and the channel are hexagonal.

7. The device of claim 1, wherein the port and the channel are approximately 25 mm in diameter.

8. The device of claim 1, wherein the channel comprises a watertight top seal and bottom seal.

9. The device of claim 8, wherein the top seal comprises a rubber gasket and the bottom seal comprises an adhesive aluminum foil seal.

10. The device of claim 8, wherein the channel comprises a buffer.

11. The device of claim 1, wherein the device further comprises a label.

12. The device of claim 1, wherein the interior surface of the collection cup is coated with RNAase inhibitor.

13. The device of claim 1, wherein the interior of the collection cup is partially coated with a colorimetric or physical indicator of the presence of exhaled aerosols.

14. An exhaled aerosol collection kit, comprising:
a device comprising: a collection cup, wherein the exterior base of the collection cup is flat and interior of the collection cup is coated with RNAase inhibitor; and a lid, wherein the lid comprises a port that forms a channel through the lid and wherein the lid is configured to attach to the collection cup;
a barcode;
a transport bag; and
a transport box.

15. The kit of claim 14, wherein the channel comprises a top seal, a bottom seal, and a buffer.

16. A method of using the kit of claim 14 by a subject, comprising the steps of:
scanning the barcode to associate the device with the subject's software account;
removing the lid from the collection cup;
holding the subject's breath for up to 15 seconds;
placing the collection cup over the subject's mouth and coughing into the collection cup at least once;
closing the device by reattaching the lid to the collection cup;
placing the device in a transport bag;
sealing the transport bag;
placing the transport bag in a transport box;
sealing the transport box; and
providing the transport box to a testing facility.

17. The method of claim 15, wherein the step of placing the collection cup over the subject's mouth further comprises spitting into the collection cup at least once.

18. The method of claim 15, wherein the subject wraps a tissue around the rim of the collection cup when performing the step of coughing into the collection cup.

19. The method of claim 15, further comprising processing the device by the testing facility, comprising the additional steps of:
removing the transport bag from the transport box;
incubating the transport bag containing the device at approximately 65° C. for approximately 30 minutes;
adding buffer to the collection cup;
sealing the lid on the collection cup;
agitating the device;
extracting buffer from the collection cup; and
subjecting the extracted buffer to PCR amplification using primers specific to at least one pathogen to form an amplicon;
sequencing the amplicon; and
uploading the sequencing results to the subject's software account.

20. The method of claim 19, wherein the sequencing results are formatted to show the presence or absence of the at least one pathogen in the subject's exhaled aerosols.


SEQUENCE LISTING (8 sequences)

SEQ ID NO: 1 - DNA, 20 bp, Homo sapiens
gaccccaaaatcagcgaaat

SEQ ID NO: 2 - DNA, 24 bp, Homo sapiens
tctggttactgccagttgaatctg

SEQ ID NO: 3 - DNA, 19 bp, Homo sapiens
ttacaaacattggccgcaa

SEQ ID NO: 4 - DNA, 18 bp, Homo sapiens
gcgcgacattccgaagaa

SEQ ID NO: 5 - DNA, 22 bp, Homo sapiens
gggagccttgaatacaccaaaa

SEQ ID NO: 6 - DNA, 21 bp, Homo sapiens
tgtagcacgattgcagcattg

SEQ ID NO: 7 - DNA, 19 bp, Homo sapiens
agatttggacctgcgagcg

SEQ ID NO: 8 - DNA, 20 bp, Homo sapiens
gagcggctgtctccacaagt